(19) United States
(12) Patent Application Publication
(10) Pub. No.: US 2010/0191022 A1
(43) Pub. Date: Jul. 29, 2010

(54) METHODS, COMPOSITIONS AND SYSTEMS RELATED TO ETHANOL MANUFACTURED FROM THE GRASS ARUNDO DONAX

(76) Inventors: Brent Alan Hawkins, Eureka, CA (US); Ben H. Altheimer, Kent, WA (US)

Correspondence Address: GRAYBEAL JACKSON LLP, 400 - 108TH AVENUE NE, SUITE 700, BELLEVUE, WA, 98004, US

(21) Appl. No.: 12/514,560

(22) PCT Filed: Nov. 13, 2007
(86) PCT No.: PCT/US07/23882

§ 371 Date: Mar. 30, 2010

Related U.S. Application Data
(60) Provisional application No. 60/858,913, filed on Nov. 13, 2006.

(51) Int. Cl. C07C 31/08; C07C 31/04; G06F 17/50

(52) U.S. Cl. 568/840; 703/1

(57) ABSTRACT

Methods, systems, compositions, etc., that use Arundo donax in a gasification process to produce ethanol with increased ethanol produced per acre of biomass, reduced input-energy needs and reduced unwanted by-products. The methods, systems, compositions, etc., are capable of producing ethanol from the sugars, starches, celluloses, hemicelluloses and lignin of the Arundo donax biomass plant directly into ethanol substantially without by-products except for an ash stream of the inorganic plant nutrients. It does so with a better efficiency and lower use of fossil fuels than traditional commercial processes. For instance the arundo-gasification process need not use any fossil fuel on an ongoing basis to provide thermal energy to conduct the gasification process.

### CROSS-REFERENCE TO RELATED APPLICATIONS

The present application claims priority from U.S. provisional patent application No. Ser. No. 60/858,913, filed 13 Nov. 2006, which is incorporated herein by reference in its entirety and for all its teachings and disclosures.

### BACKGROUND

There is an increasing demand for fuels suitable for use in internal combustion engines and other engines, preferably fuels that replace gasoline (and other fuels) obtained from crude oil and/or burn more cleanly than fuels obtained from crude oil. One of the primary sources for such replacement fuels are synthesis gas and synthesis fuels derived from corn, grain, sewage, waste or other biomass. These sources, however, have various disadvantages such as undesirable by-products such as NO₂ or sulfur-containing compounds, or acid contaminated lignin and gypsum as well as carbon dioxide, and in some cases can require as much or more input-energy for the process of transforming the feedstock into the syngas/fuel as is obtained in the resultant syngas/fuel product.

Thus, there has gone unmet a need for improved methods of generating syngas/fuel including fuels suitable for use in internal combustion engines. The present systems and methods, etc., provide these and/or other advantages.

### SUMMARY

The methods, systems, compositions, etc., herein address one or more issues arising from sustained operation of traditional biomass technologies to produce at least fuel-grade ethanol and other biofuels, for example issues related to the equipment, processes and/or chemical additives. These issues can include the ability to deploy a sustainable biomass crop technically and economically. Traditional biomass streams include distiller's grain, syrup and carbon dioxide from fermentation processes using corn; corn is the major source of ethanol biomass today. Other biomass methods include cellulosic processes which hydrolyze cellulose and hemicelluloses of the biomass plants to break them down into sugars that can then be processed with fermentation technology. The by-products from these processes include acid contaminated lignin and gypsum as well as carbon dioxide. These acid-contaminated products by themselves might be considered hazardous waste. While existing markets have been found for these by-products they return lower value and the actual size of the market of these materials may limit the ability of these conventional processes to fill the expanding markets for ethanol.

The methods, systems, compositions, etc., herein provide Arundo donax used in a gasification process to produce ethanol with reduced, and up to virtually none, of these by-products. The methods, systems, compositions, etc., are capable of producing ethanol from the sugars, starches, celluloses, hemicelluloses and lignin of the Arundo donax biomass plant directly into ethanol substantially without by-products except for an ash stream of the inorganic plant nutrients. It does so with a better efficiency and lower use of fossil fuels than traditional commercial processes. For instance the arundo-gasification process need not use any fossil fuel on an ongoing basis to provide thermal energy to conduct the gasification process.

The present systems and methods comprises systems processes, etc., for producing a predetermined C_{X}H_{Y}O_{Z} product, wherein X, Y and Z can be integers, from a primary Arundo donax feedstock comprising substantially only Arundo donax as a carbon source supplemented by a secondary feedstock comprising: providing the primary Arundo donax feedstock with a water content of not greater than about 20% to 30% and typically not more than 25%; indirectly heating the primary Arundo donax feedstock generally in the absence of oxygen to produce a gas stream and solids; cleaning the gas stream and removing CO₂ and solids to produce a cleaned gas stream; determining the amount of CO and H₂ in the cleaned gas stream; partially oxidizing the secondary feedstock to produce heat for the indirect heating of the primary Arundo donax feedstock and to produce a secondary gas stream, wherein the secondary feedstock can be selected based on determining the heat desired to indirectly heat the primary Arundo donax feedstock, comparing the percentage of CO and H₂ in the cleaned gas stream with the desired CO and H₂ to produce the predetermined C_{X}H_{Y}O_{Z} product and determining an additional amount of CO and H₂ desired from the secondary feedstock then determining the secondary feedstock, and wherein the process further comprises calculating the amount of CO, H₂ and heat to be produced from the secondary feedstock; combining the CO and H₂ from the cleaned gas stream with the secondary feedstock gas stream to produce a mixed gas stream; providing a catalyst for the mixed gas stream to produce a predetermined C_{X}H_{Y}O_{Z} gas; and distilling the predetermined C_{X}H_{Y}O_{Z} gas to produce the predetermined C_{X}H_{Y}O_{Z} product.

The process can further include the steps of determining whether CO to H₂ in the secondary gas stream meets the additional requirements of CO and H₂ for the predetermined C_{X}H_{Y}O_{Z} product, and, where additional H₂ can be desired prior to the combining step, passing CO through a water/gas shift wherein the CO can be mixed with water to produce H₂ and CO₂ and then passing the remaining CO and H₂ to the combining step, and if desired cleaning the secondary gas stream and removing CO₂ and solids to produce a cleaned gas stream. Steam can be used for the indirect heating step.

The raw primary Arundo donax feedstock can have a water content of higher than the desired percent, e.g., 25%, and the process further includes drying the raw primary Arundo donax feedstock to produce the primary feedstock. CO₂ can be removed using an amine separator and the predetermined C_{X}H_{Y}O_{Z} product can be methanol or ethanol. The catalyst can be a nickel and copper catalyst. The predetermined C_{X}H_{Y}O_{Z} gas can be methanol gas and can further include the steps of adding a second catalyst to the methanol gas to form a second predetermined C_{X}H_{Y}O_{Z} gas and distilling the second predetermined C_{X}H_{Y}O_{Z} gas to produce the predetermined C_{X}H_{Y}O_{Z} product.

The primary Arundo donax feedstock can consist essentially of Arundo donax, the secondary feedstock comprises substantially Arundo donax or consist essentially of Arundo donax. The C_{X}H_{Y}O_{Z} product can be chosen from the group consisting of alcohols, aldehydes, ketones, carboxylic acids, esters and other oxygenated hydrocarbon derivative.

In another aspect, the processes comprise designing a plant for producing a predetermined C_{X}H_{Y}O_{Z} product from a primary Arundo donax feedstock containing hydrocarbons and a secondary feedstock comprising the steps of: determining the heat required to indirectly heat the primary Arundo donax feedstock; comparing the percentage of CO and H₂ in the cleaned gas stream with the required CO and H₂ to produce the predetermined C_{X}H_{Y}O_{Z} product and determining the additional of CO and H₂ required from the secondary feedstock; determining the secondary feedstock; and calculating the amount of CO, H₂ and heat to be produced from the secondary feedstock.

The processes can further include the steps of determining whether CO to H₂ in a secondary gas stream from partial oxidation of the secondary feedstock meets the additional requirements of CO and H₂ for the predetermined C_{X}H_{Y}O_{Z} product and determining whether a water/gas shift can be required.

In a further aspect, the processes for producing saleable liquids from organic material comprising the steps of: providing organic material from Arundo donax generally in the absence of oxygen and separating it into solids, liquids and vapor; reacting the liquids, combining it with water vapor and producing a volatized gas stream; removing nitrogen dioxide from the gas stream to produce a scrubbed volatized gas stream; reacting the scrubbed volatized gas stream with water vapor to produce a combined volatized gas stream; removing carbon dioxide from the combined volatized gas stream to produce a subtracted volatized gas stream; reacting the subtracted volatized gas stream with methanol to produce an enhanced volatized gas stream; and distilling the enhanced volatized gas stream to produce ethanol. The process can further include the step of removing sulfur and chlorine from the liquid before volatizing the liquid, the scrubbed gas can be divided into two portions and one portion reacted to produce the combined volatized gas stream and the other portion can be reacted to produce the enhanced gas stream. Vapor from the organic material can be divided into at least two vapor steams and one water vapor stream can be used in the reacting step to produce volatized gas stream and the second water vapor stream can be used in the reacting step to produce combined volatized gas stream. The vapor can be split into three streams and the third water vapor stream can be reacted to produce a volatized vapor stream which can be reacted with the enhanced volatized gas stream. Nitrogen can be removed from the volatized vapor stream to produce a scrubbed volatized vapor stream and the scrubbed volatized vapor stream can be reacted with the enhanced volatized gas stream.

The Arundo donax biomass can be processed by gasifying the biomass in the presence of steam and generally in the absence of oxygen to produce a the Arundo donax biomass gas stream; removing carbon particles from the gas stream to produce a cleaned the Arundo donax biomass gas stream and water; processing carbon particles via water/gas shift to produce syngas combining cleaned the Arundo donax biomass gas stream and syngas with the enhanced volatized gas stream. In the distillation step water and hydrogen can be also produced, and the hydrogen can be combusted to provide heat to the system. Heat can be provided to the process by partially oxidizing natural gas in the presence of sub-stoichiometric oxygen and the gases from this process can be reacted with the enhanced volatized gas stream. The nitrogen dioxide can be removed by spraying the volatized gas stream with sodium hydroxide to form sodium nitrite or by spraying the volatized vapor gas stream with sodium hydroxide to form sodium nitrite.

The Arundo donax biomass gas stream, the water vapor from the biomass and the gases from the partial oxidizing of natural gas can be reacted in a fifth vessel prior to reacting with the volatized gas stream. The gas from partially oxidizing natural gas can be divided into two gas streams one gas stream being added to the fifth vessel and the other gas stream being added to the reacting step to produce the enhanced volatized gas stream.

The volatizing step can be in a first vessel which can be heated to a temperature of between 225 and 300° C. and at a pressure of between 7400 and 7600 kpa. A catalyst of cobalt can be introduced into the first vessel; the cobalt can be on a ceramic. The combining of the scrubbed volatized gas stream can be in a second vessel which can be heated to between 225 and 300° C. and a pressure of between 7400 and 7600 kpa. A second catalyst of iron and cobalt can be introduced into the second vessel, and the iron and cobalt of the second catalyst can be on a silica. The combining of the subtracted volatized gas stream can be in a third vessel which can be heated to between 350 and 380° C. and a pressure of between 19,443 and 21,490 kpa.

A third catalyst of iron and cobalt can be introduced into the third vessel and can be on a silica. The volatizing of the vapor can be in a fourth vessel which can be heated to between 225 and 300° C. and a pressure of 7400 to 7600 kpa. A fourth catalyst of iron and cobalt can be introduced into the fourth vessel and can be on a silica. The combining of the subtracted volatized gas stream can be in a fifth vessel which can be heated to between 225 and 300° C. and a pressure of between 7400 and 7600 kpa. A fifth catalyst of iron and cobalt can be introduced into the fifth vessel and can be on a silica. The gasification of the biomass can takes place at a temperature between about 650 and 900° C. Methanol or ethanol can be produced from the Arundo donax biomass. The Arundo donax biomass can be processed with the steps of: gasifying the Arundo donax biomass in the presence of steam and generally in the absence of oxygen to produce a the Arundo donax biomass gas stream; removing carbon particles from the gas stream to produce a cleaned the Arundo donax biomass gas stream and water; processing carbon particles via water/gas shift to produce syngas; and combining cleaned the Arundo donax biomass gas stream and syngas with the enhanced volatized gas stream.

These and other aspects, features and embodiments are set forth within this application, including the following Detailed Description and attached drawings. Unless expressly stated otherwise or clear from the context, all embodiments, aspects, features, etc., can be mixed and matched, combined and permuted in any desired manner. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

The figures herein are derived from US Published Patent application 20060014841, which discusses a suitable synfuel production process for the methods, systems, etc., herein.

FIG. 1 is a flow diagram of a suitable process wherein ethanol is produced.

FIG. 2 is a flow diagram of a suitable process showing the production of a general C_{X}H_{Y}O_{Z} product.

FIG. 3 is a flow diagram similar to that shown in FIG. 2 but including further processing steps in regard to a secondary feedstock.

### DETAILED DESCRIPTION

According to the methods, compositions and systems, etc., discussed herein, Arundo donax is deployed as an energy crop to provide ethanol, as well as other alcohol products such as methanol, other biofuels, etc., which are useful for, e.g., internal combustion engines. Resulting compositions include transportation fuels and gasoline additives. For example, the Arundo donax can be harvested and processed in a gasifier to produce synthesis gas (syngas). Gasification in general is a well known process, for example http://en.wikipedia.org/wiki/Gasification. In one embodiment, the syngas is reacted across a catalyst bed at desired, controlled pressure and temperature and reformed to (generally) yield ethanol and methanol. The liquids are fractionated and distilled as desired, and taken to market. A virtue of this approach is the low cost performance as compared to current feedstocks and processes. Another example of such a general gasification process applied to non-Arundo materials such as from biomass or other carbonaceous material can be found in U.S. Pat. No. 6,863,878. Although the methods, etc., herein include typical gasification processes such as those set forth in U.S. Pat. No. 6,863,878, the processes preferably are directed to more specialized processes such as those set forth in US Published Patent application 20060014841, as well as the related processes discussed in U.S. Pat. No. 6,747,067 and 6,919,488.

The methods, compositions, systems, etc., herein have a very high yield per acre of farmland for an ethanol crop. Further, the Arundo can be a superb source of biomass for processing to produce synfuels instead of using only starches, sugars and cellulose as in some current ethanol technology and the Arundo can in some embodiments provide the desired end product synfuels without use of fermentation, acid or enzymes.

Generally speaking, gasification technologies differ in many aspects but share certain general production characteristics. Typical raw materials used in gasification are coal, petroleum based materials (crude oil, high sulfur fuel oil, petroleum coke, and other refinery residuals), gases, or materials that would otherwise be disposed of as waste. The feedstock is prepared and fed to the gasifier in either dry or slurried form. The feedstock reacts in the gasifier with steam and oxygen at high temperature and pressure in a reducing (oxygen starved) atmosphere. This produces the synthesis gas, or syngas, made up primarily of carbon monoxide and hydrogen (more than 85% by volume) and smaller quantities of carbon dioxide and methane. The significant difference and advantage in the current application is the use of Arundo donax as the primary Arundo donax feedstock, which provides the substantial advantages and benefits discussed above.

Generally in gasification, the controlled temperature in the gasifier converts the inorganic materials in the feedstock (such as ash and metals) into a vitrified material resembling coarse sand. With some feedstocks, valuable metals are concentrated and recovered for reuse. The vitrified material, generally referred to as slag, is inert and has a variety of uses in the construction and building industries.

Gas treatment facilities refine the raw gas using proven commercial technologies that are an integral part of the gasification plant. Trace elements or other impurities are removed from the syngas and are either recirculated to the gasifier or recovered. Sulfur is recovered either in its elemental form or as sulfuric acid, both marketable commodities. If the syngas is to be used to produce electricity, it is typically used as a fuel in an integrated gasification combined cycle (IGCC) power generation configuration. The syngas can also be processed to produce a wide range of products, fuels, chemicals, fertilizer or industrial gases.

Four types of gasifier that can be suitable for the methods, systems, etc., herein, include counter-current fixed bed, co-current fixed bed, fluidized bed and entrained flow. Generally the counter-current fixed bed (“up draft”) gasifier consists of a fixed bed of carbonaceous fuel (e.g. coal or biomass) through which the “gasification agent” (steam, oxygen and/or air) flows in counter-current configuration. The ash is either removed dry or as a slag. The slagging gasifiers require a higher ratio of steam and oxygen to carbon in order to reach temperatures higher than the ash fusion temperature. The nature of the gasifier means that the fuel must have high mechanical strength and must be non-caking so that it will form a permeable bed, although recent developments have reduced these restrictions to some extent. The throughput for this type of gasifier is relatively low. Thermal efficiency is high as the gas exit temperatures are relatively low. However, this means that tar and methane production is significant at typical operation temperatures, so product gas must be extensively cleaned before use or recycled to the reactor. The co-current fixed bed (“down draft”) gasifier is similar to the counter-current type, but the gasification agent gas flows in co-current configuration with the fuel (downwards, hence the name “down draft gasifier”). Heat needs to be added to the upper part of the bed, either by combusting small amounts of the fuel or from external heat sources. The produced gas leaves the gasifier at a high temperature, and most of this heat is often transferred to the gasification agent added in the top of the bed, resulting in an energy efficiency on level with the counter-current type. Since all tars must pass through a hot bed of char in this configuration, tar levels are much lower than the counter-current type. In the fluidized bed gasifier, the fuel is fluidized in oxygen and steam or air. The ash is removed dry or as heavy agglomerates that defluidize. The temperatures are relatively low in dry ash gasifiers, so the fuel must be highly reactive; low-grade coals are particularly suitable. The agglomerating gasifiers have slightly higher temperatures, and are suitable for higher rank coals. Fuel throughput is higher than for the fixed bed, but not as high as for the entrained flow gasifier. The conversion efficiency can be rather low due to elutriation of carbonaceous material. Recycle or subsequent combustion of solids can be used to increase conversion. Fluidized bed gasifiers are most useful for fuels that form highly corrosive ash that would damage the walls of slagging gasifiers. Biomass fuels generally contain high levels of corrosive ash. In the entrained flow gasifier a dry pulverized solid, an atomized liquid fuel or a fuel slurry is gasified with oxygen (much less frequent: air) in co-current flow. The gasification reactions take place in a dense cloud of very fine particles. Most coals are suitable for this type of gasifier because of the high operating temperatures and because the coal particles are well separated from one another. The high temperatures and pressures also mean that a higher throughput can be achieved, however thermal efficiency is somewhat lower as the gas must be cooled before it can be cleaned with existing technology. The high temperatures also mean that tar and methane are not present in the product gas; however the oxygen requirement is higher than for the other types of gasifiers. All entrained flow gasifiers remove the major part of the ash as a slag as the operating temperature is well above the ash fusion temperature. A smaller fraction of the ash is produced either as a very fine dry fly ash or as a black colored fly ash slurry. Some fuels, in particular certain types of biomasses, can form slag that is corrosive for ceramic inner walls that serve to protect the gasifier outer wall. However some entrained bed type of gasifiers do not possess a ceramic inner wall but have an inner water or steam cooled wall covered with partially solidified slag. These types of gasifiers do not suffer from corrosive slags. Some fuels have ashes with very high ash fusion temperatures. In this case mostly limestone is mixed with the fuel prior to gasification. Addition of a little limestone will usually suffice for the lowering the fusion temperatures. The fuel particles must be much smaller than for other types of gasifiers. This means the fuel must be pulverized, which requires somewhat more energy than for the other types of gasifiers. By far the most energy consumption related to entrained bed gasification is not the milling of the fuel but the production of oxygen used for the gasification.

Turning to the Figures, the figures herein are derived from US Published Patent application 20060014841, which discusses a suitable synfuel production process for the methods, systems, etc., herein. The 20060014841 application discusses production of synfuels from feedstock such as biomass such as cellulosic plant materials, processed cellulosic products, animal or human excrement, processed animal or human sewage, fossil fuels of any type, plant oils, and other feedstock containing hydrocarbons, but does not teach nor suggest either the use of Arundo donax as a feedstock nor the advantages of Arundo donax as a feedstock. The Arundo donax is provided the primary feedstock in the processes below and can optionally also be the secondary feedstock, or both.

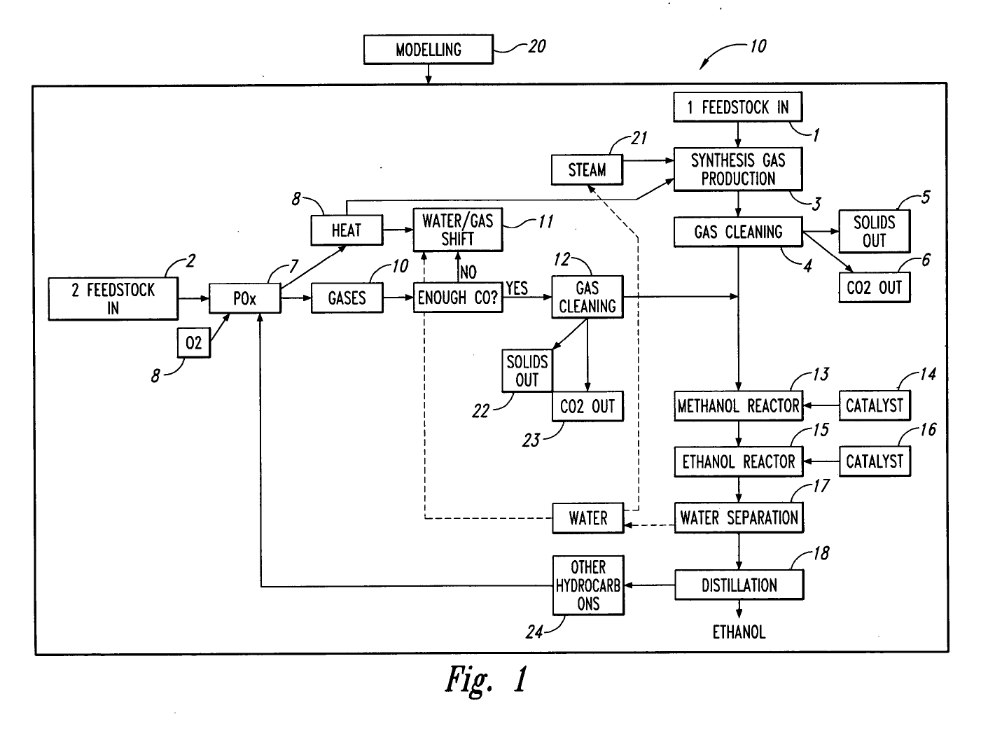

In FIG. 1, a computer modeling process 20 begins with an analysis of a primary Arundo donax feedstock comprised substantially of, and may consist essentially of, Arundo donax. In certain circumstances, the feedstock may also comprise smaller amounts of, e.g., wood residues from harvesting, milling or municipal activities, pulp and paper bark or sawdust, human or animal sewage, or high protein residues from fermentation of grains often referred to as dry distiller's grains or dry distiller's grains and solubles. Chemical analysis and a metal analysis (such as ICAP) can be helpful to obtain information on, e.g., carbon, hydrogen, oxygen and nitrogen content, BTU value, water content. In comparison to the 20060014841 application, where such analysis is required due to the variable nature of the feedstock, in the current methods such pre-analysis is only desired in certain circumstances such as where non-Arundo feedstocks are provided with the substantially only Arundo feedstock, and indeed if the source of the Arundo feedstock is well known and/or reliable, the pre-testing can be omitted completely.

The basic process steps include gasification of the primary Arundo donax feedstock to produce a synthesis gas, gas cleaning, blending the gas stream with another cleaned gas stream produced by the partial oxidation of a secondary feedstock that is typically also substantially only Arundo donax, which secondary feedstock may or may not have been subjected to a water/gas shift to adjust the carbon monoxide content, then reacting the combined synthesis gases to produce methanol first, then converting the methanol to ethanol.

The process design is thus begun using computer software. In the model, the primary Arundo donax feedstock is gasified using indirect heat in the absence of oxygen/air, to produce a primary synthesis gas of carbon monoxide and hydrogen. Also present in the gases will be particulate matter (carbon, or ash) and carbon dioxide. There may also be other hydrocarbon gases such as methane (CH₄). The information of the breakdown of the feedstock can be derived from experience and literature, and can provide the basis for the mass balance of the process. Because other products of gasification are likely to be produced, values for carbon dioxide, methane and small hydrocarbons such as propane and ethane are also fed into the computer simulation model.

The modeling process can be iterative. As each step in the input and processing are achieved, the information from the model is evaluated. Adjustments to the volumes of input are made in the model, as are variants in energy supplied for the gasification, volume and temperature of the steam supplied, and gas velocities through the process.

The desired product, in this case ethanol, is then selected. Currently, other products which can alternatively be made are derivatives of methanol, and include formaldehyde (CHOH) and acetic acid (CH₃COOH). The carbon monoxide and hydrogen ratio obtained from the gasification of the primary Arundo donax feedstock are measured in the present invention against the ideal amounts of carbon, oxygen and hydrogen needed to form ethanol. A secondary Arundo donax feedstock is then contemplated, in consideration of its carbon content, BTU value, availability and cost. It can be selected on the basis of the following questions: 1. How much heat is needed to achieve the gasification of the primary Arundo donax feedstock? 2. How much additional carbon monoxide and hydrogen are required to supplement the primary synthesis gas? 3. Can the gases be obtained from partial oxidation of the secondary feedstock? 4. Will the gas mixture then require further processing by a water/gas shift?

The selection of the secondary feedstock is typically Arundo donax but could be a gas, liquid or solid hydrocarbon. Examples of gases include natural gas, landfill gas, propane or butane; examples of liquids include gasoline, diesel fuel, bio-diesel (defined as diesel fuel make from biomass), bio-oil (defined as plant oils produced by reprocessing or from the pyrolysis of wood); examples of solids include wood or any type, crop residues, organic wastes, paper waste, plastics. Once a material is selected, particularly if a feedstock other than Arundo is selected since Arundo donax would be well-known to the user, information on the gases which are produced from its partial oxidation are input into the computer model. From evaluation of the gas composition after oxidation, a decision is made to further process the gases using a water/gas shift. The process of the present invention converts carbon monoxide to carbon dioxide, thereby adjusting the final carbon monoxide ratio, a step which may not always be necessary. The gases are then combined with the primary gas stream, and the model can then determine the output of methanol firstly, and then the final ethanol output.

The nature of the computer software allows changes to any input, which can then be adjusted manually or automatically until the process flow is satisfactory. That typically means that as much ethanol is produced from the inputs as possible, as little carbon monoxide is exhausted from the process, and as little volume of unwanted hydrocarbons from the catalytic steps are recycled into the initial gasification or secondary partial oxidation steps.

An exemplary embodiment of the physical process is described as follows:

In FIG. 1, generally at 10, the primary Arundo donax feedstock 1 is prepared by whatever means necessary, such as chipping, grinding, chopping and drying to achieve a moisture content of typically 25%, and a size of 2″ or less in any direction; other sizes and moisture contents can be used as desired. The feedstock is fed into the indirectly-heated gasifier 3 in the absence of air or oxygen, and gasified using steam 21 or other heat source as a fluidizing medium. The gas stream evolved is cleaned 4 and solids 5 and carbon dioxide 6 removed. If only Arundo donax is used as the feedstock, the solids may consist only of ash or minerals such as sodium, nitrogen compounds, potassium, copper, silica, phosphorus. The exact mixture is of course determined by the composition of the primary Arundo donax feedstock. The carbon dioxide is typically scrubbed out of the gas stream using an amine separator, but other methods can be used. The economics of the process will typically determine which method is utilized.

A secondary, typically Arundo donax, feedstock 2 is prepared by whatever means necessary, such as chipping, grinding, chopping and drying to achieve a moisture content of not more than 25%, and a size of 2″ in any direction; other sizes and moisture contents can be used as desired. The feedstock is then partially oxidized in a gasifier 7 using oxygen 8 which can be supplied for example, from an oxygen generator or molecular sieve. In the event the secondary feedstock is a liquid or a gas, the feedstock can be fed into a burner and partially oxidized, using oxygen 8, supplied from either source. The heat produced from this step is used to heat the gasifier 3 to gasify the primary Arundo donax feedstock, and, if desired, the water/gas shift reactor 11. The gases produced from the partial oxidation process are either cleaned 12 or sent to the water/gas shift reactor 11. Gases emerging from the water/gas shift reactor 11 are cleaned 12 and solids 22 and carbon dioxide 23 removed. The solids are again determined by the chemical composition of the secondary feedstock, but will be low volume if a gas or liquid is utilized. The carbon dioxide is scrubbed out using whatever method is desired, usually the most economical, as with the cleaning of gases in the primary step.

Gases from the cleaning process 12 are merged with the primary gases emerging from the gas cleaning sequence 4. The merged gas stream is sent to the methanol reactor 13 which is supplied with catalyst 14. The catalyst can be nickel and copper generally in the proportions of 93:7; suitable catalysts are well known. The product methanol and any other hydrocarbons formed in the reactor 13 are sent to the ethanol reaction process 15. The catalyst 16 is used to convert the methanol to ethanol and is generally a nickel copper catalyst in the proportions of 75:25; again, suitable catalysts are well known. The process may be reactive distillation, in which the methanol is converted to methyl acetate, then split into ethanol and methanol. In this case, the methanol is continuously recycled through to be reprocessed.

The products of the ethanol reaction step 15 are sent to a water separator 17 and the water is recycled into the system for use in the water/gas shift reactor 11 or the steam generator 21 to supply steam for the gasifier. The products are then sent to distillation 18, in which the ethanol is purified. Any other hydrocarbon liquids 24 removed in the distillation are sent to the partial oxidation process 7 or to the primary gasifier 3.

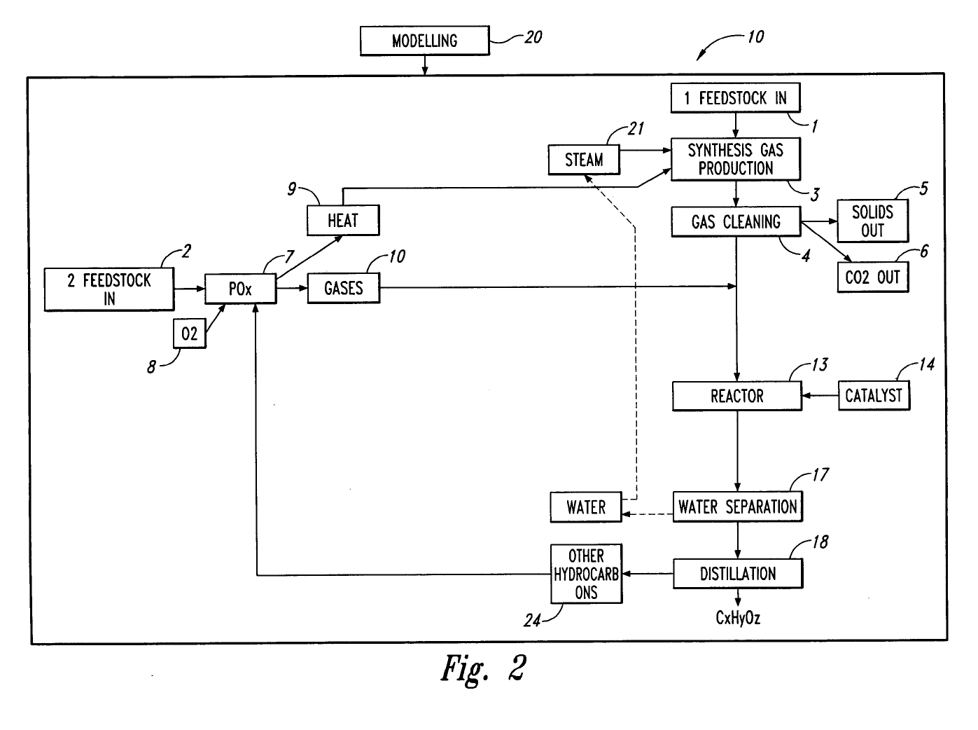

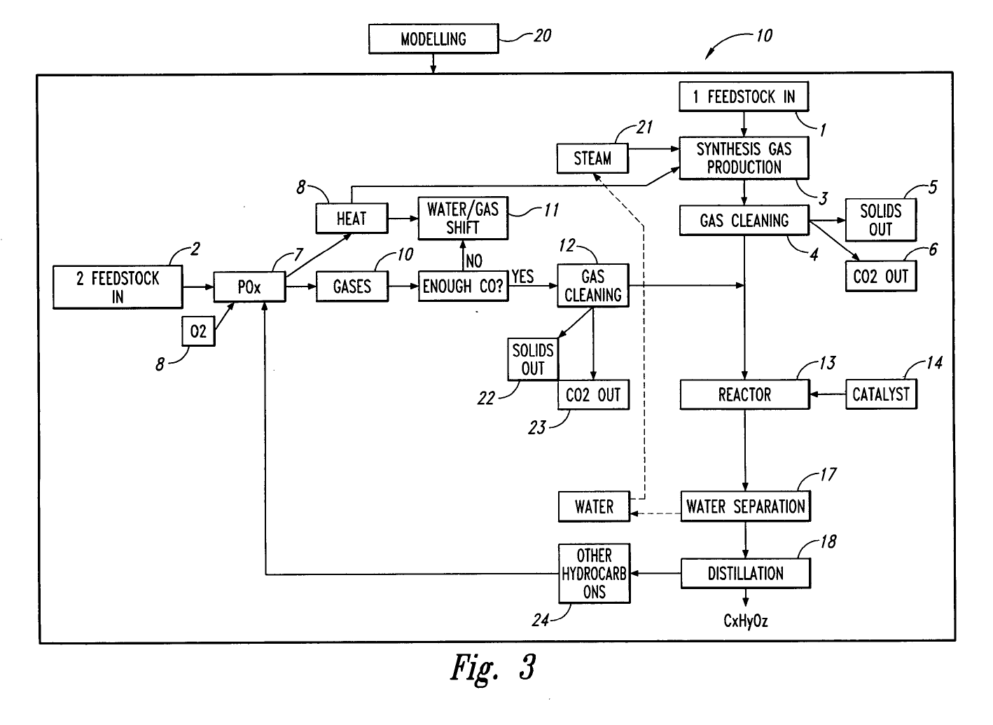

It will be appreciated that the above description is in regard to a specific example. The methods, etc., can be described in more general terms and these are shown in FIGS. 2 and 3. For example, in FIG. 2 the example is for the instance where the secondary feed stock 2 is a predetermined gas such that certain steps would not be required. Specifically the water/gas shift step and the gas cleaning step would not be required. Further, with the advent of new catalysts it may be possible to go directly to the desired C_{X}H_{Y}O_{Z} product without going through methanol. Similarly FIG. 3 is similar to both FIGS. 1 and 2 in that it includes the water/gas shift and the gas cleaning steps of FIG. 1 but it contemplates other catalyst than FIG. 2.

The range of C_{X}H_{Y}O_{Z} products which can be formed from synthesis gas are limited only by the availability of catalysts to do so. Certain ones have been available for many years, such as the Fischer-Tropsch group, which are used heavily to produce fuels and chemicals from the synthesis gas generated from the gasification of coal. There are many other catalysts designed to perform specific reactions such as the conversion synthesis gas to methanol. The challenge is to fit the catalyst to the desired product with the most effective use of the synthesis gas produced.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “means” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

What is claimed is:

1. A process for producing a predetermined C_{X}H_{Y}O_{Z} product, wherein X, Y and Z are integers, from a primary Arundo donax feedstock comprising substantially only Arundo donax as a carbon source supplemented by a secondary feedstock comprising: providing the primary Arundo donax feedstock with a water content of not greater than about 25%; indirectly heating the primary Arundo donax feedstock generally in the absence of oxygen to produce a gas stream and solids; cleaning the gas stream and removing CO₂ and solids to produce a cleaned gas stream; determining the amount of CO and H₂ in the cleaned gas stream; partially oxidizing the secondary feedstock to produce heat for the indirect heating of the primary Arundo donax feedstock and to produce a secondary gas stream, wherein the secondary feedstock is selected based on determining the heat desired to indirectly heat the primary Arundo donax feedstock, comparing the percentage of CO and H₂ in the cleaned gas stream with the desired CO and H₂ to produce the predetermined C_{X}H_{Y}O_{Z} product and determining an additional amount of CO and H₂ desired from the secondary feedstock then determining the secondary feedstock, and wherein the process further comprises calculating the amount of CO, H₂ and heat to be produced from the secondary feedstock; combining the CO and H₂ from the cleaned gas stream with the secondary feedstock gas stream to produce a mixed gas stream; providing a catalyst for the mixed gas stream to produce a predetermined C_{X}H_{Y}O_{Z} gas; and distilling the predetermined C_{X}H_{Y}O_{Z} gas to produce the predetermined C_{X}H_{Y}O_{Z} product.

2. A process as claimed in claim 1 further including can further include determining whether CO to H₂ in the secondary gas stream meets the additional requirements of CO and H₂ for the predetermined C_{X}H_{Y}O_{Z} product, and, where additional H₂ is desired prior to the combining step, passing CO through a water/gas shift wherein the CO is mixed with water to produce H₂ and CO₂ and then passing the remaining CO and H₂ to the combining step.

3. A process as claimed in claim 2 further including the step of cleaning the secondary gas stream and removing CO₂ and solids to produce a cleaned gas stream.

4. A process as claimed in claim 3 wherein steam is used for the indirect heating step.

5. A process as claimed in claim 3 wherein a raw primary Arundo donax feedstock has a water content of higher than 25% and the process further includes drying the raw primary Arundo donax feedstock to produce the primary feedstock.

6. A process as claimed in claim 3 wherein the CO₂ is removed using an amine separator.

7. A process as claimed in claim 3 wherein the predetermined C_{X}H_{Y}O_{Z} product is methanol.

8. A process as claimed in claim 7 wherein the catalyst is a nickel and copper catalyst.

9. A process as claimed in claim 3 wherein the predetermined C_{X}H_{Y}O_{Z} gas is methanol gas and further including can further include adding a second catalyst to the methanol gas to form a second predetermined C_{X}H_{Y}O_{Z} gas and distilling the second predetermined C_{X}H_{Y}O_{Z} gas to produce the predetermined C_{X}H_{Y}O_{Z} product.

10. A process as claimed in claim 9 wherein the predetermined C_{X}H_{Y}O_{Z} product is ethanol.

11. A process as claimed in claim 1 wherein the primary Arundo donax feedstock consists essentially of Arundo donax.

12. A process as claimed in claim 3 wherein the secondary feedstock comprises substantially Arundo donax.

13. A process as claimed in claim 12 wherein the secondary feedstock consists essentially of Arundo donax.

14. A process as claimed in claim 1 wherein the C_{X}H_{Y}O_{Z} product is chosen from the group consisting of alcohols, aldehydes, ketones, carboxylic acids, esters and other oxygenated hydrocarbon derivative.

15. A process for designing a plant for producing a predetermined C_{X}H_{Y}O_{Z} product from a primary Arundo donax feedstock containing hydrocarbons and a secondary feedstock comprising the steps of: determining the heat required to indirectly heat the primary Arundo donax feedstock; comparing the percentage of CO and H₂ in the cleaned gas stream with the required CO and H₂ to produce the predetermined C_{X}H_{Y}O_{Z} product and determining the additional of CO and H₂ required from the secondary feedstock; determining the secondary feedstock; and calculating the amount of CO, H₂ and heat to be produced from the secondary feedstock.

16. A process as claimed in claim 15 further including can further include determining whether CO to H₂ in a secondary gas stream from partial oxidation of the secondary feedstock meets the additional requirements of CO and H₂ for the predetermined C_{X}H_{Y}O_{Z} product and determining whether a water/gas shift is required.

17. A process as claimed in claim 15 wherein the C_{X}H_{Y}O_{Z} product is chosen from the group consisting of alcohols, aldehydes, ketones, carboxylic acids, esters and other oxygenated hydrocarbon derivative.
